(19) United States
(12) Patent Application Publication
(10) Pub. No.: US 2004/0142012 A1
(43) Pub. Date: Jul. 22, 2004

(54) Geometry retainable devices for body cavities

(76) Inventors: Craig Robert Bunt, Auckland (NZ); Michael John Rathbone, Auckland (NZ); Colin Roger Ogle, Auckland (AU); Mark Andrew Wyllie, New Zealand (NZ)

Correspondence Address: JACOBSON HOLMAN PLLC, 400 SEVENTH STREET N.W., SUITE 600, WASHINGTON, DC, 20004, US

(21) Appl. No.: 10/470,735

(22) PCT Filed: Feb. 1, 2002
(86) PCT No.: PCT/NZ02/00011

(30) Foreign Application Priority Data
Feb. 9, 2001 (NZ) 509894

(51) Int. Cl. A61F 2/00

(52) U.S. Cl. 424/423

(57) ABSTRACT

A passive or active release device for a medicament or other substance for a target species mammal. The device is of a kind capable of being inserted into a body cavity of a target species mammal and deploying resiliently to one or more retention condition(s). The device is wholly or in part moulded from poly (-caprolactone) (optionally impregnated with at least a physiologically effective agent to be released therefrom or optionally to carry some device or material to be body cavity retained at least for a period). The device is preferably of substantially a “T” or “Y” shape.

### TECHNICAL BACKGROUND

The present invention relates to geometry retainable devices for body cavities and particularly although not solely to such devices for intra vaginal retention.

Both passive release and active release devices are known for insertion and retention in body cavities of appropriate mammals. In this respect reference is made to our New Zealand Patent No. 286492 which relates to a passive release device suitable for intra vaginal insertion, retention and withdrawal from a target species mammal (for example, a cow). Such a device is preferably in the form of a silicone rubber surround of a resilient nylon spine which provides the appropriate resilience with the silicone rubber surround by virtue of its impregnation with suitable substantially homogeneous quantities of progesterone being useful in the synchronisation of animal oestrous within a herd where such devices are utilised.

### BACKGROUND ART

An equivalent type device useful for insertion in pigs is that disclosed in our New Zealand Patent No. 314937/329228 for pigs.

Again such a device is a spined article having a silicone rubber surround appropriately impregnated so as to act as a passive release device intra vaginally.

An active delivery device is such as disclosed or described (with reference to prior art also) in PCT/NZ99/00083 (published as WO 99/65497) where again the retention is preferably by a wing like dependance similar to that disclosed in respect of the aforementioned passive release devices still other active release devices includes that of our New Zealand Patent No. 329338 which includes a body with a multi barrel active delivery feature, such a device, if being used intra vaginally again being retainable by appropriate geometry retention means.

Still another type of material delivery device is that disclosed in PCT/NZ99/00126 (published as WO 00/09037). This discloses an active release device as an alternative to that of WO 99/65497. This particular device relies upon electrolysis gases being issued at one or more electrode within a matrix which includes both the active agent and a carrier thereby expressing progressively more of the matrix to the physiological environment the more gas that is generated. Such devices are capable of being intra vaginally or otherwise retained in a manner similar to the other devices described herein or referred to.

Such devices need not however depend upon an active agent impregnated matrix of a silicone rubber, for example, our New Zealand Patent No. 329229/330595 and our PCT/NZ99/00070 (published as WO 99/63967) discloses the use of a matrix forming material having a biodegradable characteristic after withdrawal from use, such a matrix being of either a poly (ε-caprolactone) material or a starch like polysaccharide impregnated with the appropriate active agent (usually progesterone, preferably in the presence of a suitable cyclodextrin).

The full content of all of the aforementioned patent specifications is hereby here included by way of reference.

A feature of such retention devices is their capability of being safely retained within an animal for any desired retention period yet to be withdrawable (if it is to be withdrawn) all without discomfort of significance or tissue damage to the recipient mammal.

The present invention recognises that surprisingly an intra vaginal device formed solely of poly (ε-caprolactone), poly (ε-caprolactone) impregnated with a suitable progesterone or poly (ε-caprolactone) impregnated with both a suitable progesterone and a suitable cyclodextrin can be formed to define a body and dependent wings, fingers or the like (hereafter “wings”) where despite the overall device in its preferred form being limp to a substantial extent can have nonetheless an adequate retention performance with reduced animal trauma where the wings are connected to the body through a region of the moulding of greater bulk (at least in one plane) than the more distal regions of the wings (at least in the same plane being preferably that of resilient movement between the insertion and retention conditions and/or the retention and withdrawal conditions).

Indeed we have also determined that a suitable material (eg; a poly (ε-caprolactone) or a starch-like polysacchride) having a better resilience memory than nylon yet not substantially affected by physiological conditions can be utilised for the provision of an intra vaginally retainable device (whether it itself is to be impregnated, coated or the like to provide passive release or is simply to carry some release device (active or passive)) where the distal region of such devices retention wings are more slight in at least axes of required resilience to accommodate insertion, retention and withdrawal than does a nodal region which connects said distal regions to the body even if the overall impression of the body is that that it is itself limp.

### DISCLOSURE OF THE INVENTION

It is therefore an object of the present invention to provide an intra vaginal device or variations of such a device suitable for body cavity retention.

In one aspect the present invention is an intra vaginally retainable device which whilst at least in part limp in character is capable of being inserted in an insertion condition into the vaginal tract of a target species mammal, deploying resiliently to one or more retention condition(s) in such tract from the insertion condition and (if desired) being withdrawn from such tract, said device (preferably moulded) having

an elongate body, and

at least two wings capable of deploying to extend more outwardly from longitudinal axis of the body the constrained for insertion condition,

wherein said wings and said body have been formed from a material (impregnated or otherwise) having a resilient memory greater than that of nylon and which is substantially unaffected by the physiological conditions of the target species during such retention.

Preferably said material has been impregnated with either a progesterone or a progesterone and clyclodextrin(s).

Preferably said body is limp and said wings are limp and each is solely of said material (impregnated or otherwise) i.e. absent any spine or like structure of any other material.

Preferably said material is a poly (ε-caprolactone) or a poly (ε-caprolactone) based matrix.

Preferably the device has a substantially “T” (upper or lower case, i.e. “T” or “t”) or “Y” (upper or lower case) configuration or a variant thereof which may include additional wings.

Preferably the body (i.e. the stem of the substantially “T” or substantially “Y” configuration) is substantially limp from its nodal integrally moulded connection to its wings.

Preferably said wings, when viewed in a direction that displays said substantially “T” or substantially “Y” configuration, are less bulky in their distal regions than their nodal region, (ie; their regions at and/or adjacent said nodal integrally moulded connection with said body).

In another aspect the invention is an intravaginal device moulded in an at least progesterone impregnated poly (ε-caprolactone) material to define substantially a “T” (upper or lower case) shape or substantially a “Y” (upper or lower case) shape,

wherein said body defined by the stem of the shape is limp and at least the distal region of each arm of the shape is limp,

and wherein the poly (ε-caprolactone) material contains from 0.1 to 3 grams progesterone and the moulded material contains from 5 to 70% w/w progesterone and from 0 to 70% cyclodextrin,

and wherein the moulded surface is from 15 to 200 cm².

Preferably each arm has a distal region extending outwardly from a curved proximal region that outstands from the stem.

Preferably the curved proximal region of one arm is similar to that of the other and each curve is in plane in which its distal regions has a favoured freedom of resilient deformation.

Preferably said curve is an integral “U” shape at one end of the stem, from which U shape each arm continues integrally to extend outwardly of the stem axis thereby to define substantially an upper case “T” shape with the stem, the “U” region being a nodal zone of the retention zone of each wing from the stem.

Preferably the stem is from 50 to 150 mms in length.

In another aspect the invention is an intra vaginal device of a moulded matrix which includes a material capable of having or having a resilience memory better than that of nylon, said device having an elongate limp body from which at least a pair of retention wings depend via a nodal region, said nodal region controlling the planar flexure of proximate regions of each wing more strongly than the planar flexure of distal regions of each wing, such planar flexure being in a plan that can include substantially all of the elongate body.

Preferably, in a relaxed condition with said body substantially straight the angle to the distal ends of each wing from the longitudinal axis of the body from the non nodal region end is less than 150 degrees.

Preferably intra vaginally in a retention condition said angle is in the range of from 90 to 180 degrees.

Preferably the length of the body is from 50 to 150 mms.

Preferably the device is at least in part moulded matrix of said material which includes therein both a cyclodextrin and an intra vaginally effective active agent.

Preferably said matrix is at least in part (and preferably primarily) of a polymer or a mixture thereof.

Preferably said poly is poly (ε-caprolactone). As an alternative of said polymer is a starch-like polysaccharide.

Preferably the cyclodextrin(s) comprise from 5 to 70% w/w.

Preferably the active agent(s) comprise from 5 to 70% w/w.

Preferably agent is progesterone in the concentration of 5 to 70% w/w.

Preferably the cyclodextrin is hydroxypropyl β-cyclodextrin in the concentration of 5 to 70% w/w.

Preferably devices are capable of achieving with an animal (or group of animals) a blood serum level of greater than 2ng/ml for a period of at least 5 days of progesterone solely as a result of inserting and retaining in the or each animal for at least the 5 day period a device of any of the kinds of devices of the present invention.

Preferably said device has a loading of from 0.1 to 3 gms of progesterone for the target animals such as cattle, sheep, goats, deer, etc.

Preferably said device has an impregnated matrix surface of from 15 to 200 cm².

In another aspect the present invention consists in an intra vaginally retainable device which whilst largely limp in its character is capable of being inserted into the vaginal tract of a target species mammal, deploying resiliently to one or more retention condition(s) in such tract and (if desired) being withdrawn from such tract, said device having at least two wings extending more outwardly from the vaginal tract axis than in the insertion condition, said wings and said body being formed from a material (preferably impregnated or otherwise with an appropriate agent, e.g. a suitable progesterone or a suitable progesterone and a suitable clyclodextrin), having a resilient memory greater than that of nylon and being substantially unaffected by the physiological conditions of the target species during such retention.

Preferably said device is moulded in poly (ε-caprolactone) or a poly (ε-caprolactone) based matrix.

In another aspect the device is moulded in a starch-like polysaccharide and is at least in part encased with a water impermeable layer.

Preferably said device is a device having a substantially “T” or “Y” configuration or a variant thereof which may include additional wings.

Preferably the body (i.e. the stem of the substantially “T” or substantially “Y” configuration) is substantially limp from its nodal connection to its wings.

Preferably said wings when viewed in a direction that displays said substantially “T” or substantially “Y” configuration are less bulky in their distal regions than their nodal region, ie; their regions at and/or adjacent said nodal interconnection with said body.

Preferably the device is a device also of any of the kinds hereinafter described.

In a first aspect the invention is an intra vaginal device of a kind capable (at least) of

(i) being inserted into the vaginal tract of a target species mammal, and

(ii) deploying resiliently to one or more retention condition(s) in such tract,

wherein in said retention condition(s) at least two wings will extend more outwardly from the vaginal tract axis than in the insertion condition,

and wherein the wings and at least part of the body of the device from which the wings depend is moulded in a poly (ε-caprolactone) material,

and wherein said wings are integral with said at least part of the body of the device through a nodal transition of greater rigidity owing to bulk than the distal region(s) of each wing.

In an alternative form said poly (ε-caprolactone) material may be substituted for by a suitable starch-like polysacchride or some other material having a better resilience memory than nylon yet which is preferably less affected by intra vaginal tract physiological conditions than nylon.

Preferably said device whether as aforesaid or hereafter described is for the purpose of suppressing oestrus so as to allow for an individual target species mammal or for a herd of such target species mammals the onset of oestrus shortly after the withdrawal of such a device or such devices.

In respect of the performance of such procedures reference is made back to the aforementioned patent specifications, the full content of which is hereby here included by way of reference.

In a further aspect the present invention consists in an intra vaginal device of a kind capable of being inserted into the vaginal tract of a target species mammal, deploying resiliently to one or more retention condition(s) in such tract, and (if desired) being withdrawn from such tract,

wherein at least two wings depend from at least part of the body of the device, said wings and said at least part of the body of the device being a unitary moulding of a suitable resilient material (optionally coated in order to minimise physiological effects on the integrity of such material during vaginal tract retention),

and wherein the moulded form of said wings and at least part of the body is such that a more distal region of the wings is more flexible in bending towards one or both of the vaginal tract axis and the body axis (if any) than the “nodal region” of said wings with said at least part of the body.

Preferably said material from which the moulded region aforesaid is prepared is of a mouldable material having a better memory than nylon yet which is less affected by intra vaginal tract physiological conditions than nylon as far as its integrity and/or resilience is concerned.

Preferably said material is a suitable poly (ε-caprolactone) material.

Preferably said device is substantially “T” or “Y” shaped with a nodal region which includes the body proximate regions of each wing and the wing proximate region of at least part of the body (i.e. the body being the stem of the “T” or “Y”), such node whilst of a similar (if not identical) material to the distal region of each wing being less flexible owing to greater moulded bulk.

Preferably the distal region of the wings is thinner in at least a plane which better enables bending in either rotational sense relative to the vaginal tract axis and/or a substantially “T” or “Y” stem defined body of the device.

Preferably the body of the device is fully moulded.

Preferably said material is poly (ε-caprolactone) impregnated with a material to be released, (e.g. for example a progesterone material or progesterone and clyclodextrin materials).

Preferably said device has any of the configurations hereinafter described with reference to any one or more of the accompanying drawings.

In a further aspect the present invention consists in an intra vaginal device of a kind capable of being inserted into the vaginal tract of a target species mammal, deploying resiliently to one or more retention condition(s) in such tract to release progesterone, and being withdrawn from such tract,

wherein said retention condition(s) at least two wings extend more outwardly from the vaginal tract axis than in the insertion and withdrawal conditions,

wherein the wings and the body of the device from which the wings depend is moulded in poly (ε-caprolactone) impregnated with progesterone(s) (and optionally also cyclodextrin(s)) to a substantially limp elongate body form bulkily (at least in one plane) connected to the wings at the nodal region with the wings rendered more flexible (preferably in that plane) in their non-nodal region or regions to better conform to the wall of the vaginal tract.

In still another aspect the present invention consists in an intra vaginal device moulded to substantially a “T” or substantially “Y” configuration where the wings (those parts that extend outwardly from the stem of the substantially “T” or substantially “Y” configuration) each have the nodal region (ie; the transition from the stem to the wings) more bulky in a plane than is the distal region of each wing, thereby to ensure as far as flexibility of each wing in total is concerned greater flexibility over outer regions of each wing than at the nodal region.

Preferably said device is of a form where the transition from the stem to the wings is a substantially “U” form blended into “T” wings, e.g. substantially as hereinafter described.

Preferably said device has a wing span of about 150 mm (e.g. from 125 to 175 mm).

Preferably each wing is wider (i.e. of greater dimension in a plane normal to the plane of the “T” or substantially “r” form) than it is in the plane of the “T”.

Preferably said substantially “T” or substantially “Y” form has a body (i.e. the stem) that is substantially of a similar configuration and preferably a similar flexibility to the distal region or regions of each wing (at least away from the nodal region).

Preferably the device is as substantially as hereinafter described.

Variants of the device made of another material within the ambit of the present invention is also included.

In still a further aspect the present invention consists in a passive or active release device for a medicament or other substance for a target species mammal, said device comprising a device of a kind capable of being inserted into a body cavity of a target species mammal and deploying resiliently to one or more retention condition(s) wherein said device is wholly or in part moulded from poly (ε-caprolactone) (optionally impregnated with at least a physiologically effective agent to be released there from or optionally to carry some device or material to be body cavity retained at least for a period) and wherein the device in its moulded or moulded and optional assembled form has a plurality of wings (ie; two or more) each wing connected to the other through a nodal region and each also connected to at least part of the body of the device by the same nodal region, such nodal region owing to its bulk in section in at least one plane (poly (ε-caprolactone) alone or impregnated poly (ε-caprolactone)) having less flexibility than more distal regions or a more distal region of the wings or each wing.

Preferably the flexibility of the distal region or regions is further enhanced by (i) the asymmetry of the cross-section of such distal region or regions of the wings and/or (ii) the asymmetry of the cross-section of the nodal region.

Whilst reference is made hereto to distal regions of the wings as being more flexible than a nodal region or an inner region of each wing adjacent said body preferably there is no difference in the characteristic to the matrix (ie; material or material and its content material(s)) forming the device at such nodal region and the wings and optionally the asymmetry and/or less bulky form can be sharply defined or progressive or gradual (whether monotonically decreasing or otherwise) or some hybrid thereof.

In still a further aspect the present invention consists in a method of retaining a physiologically effective sensor and/or physiologically effective agent delivery device in a body cavity of a target species mammal (for example, any of the target species mammals referred to any of the aforementioned patent specifications) where the retention characteristics are those herein described that differ from those disclosed in the aforementioned prior art specifications.

In still a further aspect the present invention consists in retention methodology substantially as herein described with reference to any one or more of the accompanying drawings.

In other aspects the present invention consists in the use (preferably for herd oestrus synchrony purposes) of devices of the present invention.

As used herein the term “substantially “T” or “Y” shaped” includes within its ambit any hybrid of a “T” or “Y” shape such as for example the substantially “T” or substantially “Y” shape of the device hereinafter described with reference to the accompanying drawings. The reference also, where it allows, includes forms having more than two wings, e.g. three or more, where they deploy from a stem such as that of a “T” or “Y” but are asymmetrically or symmetrically disposed around the axis of any such stem defined body or body part.

### DETAILED DESCRIPTION OF THE INVENTION

Preferred forms of the present invention will now be described with reference to the accompanying drawings in which:

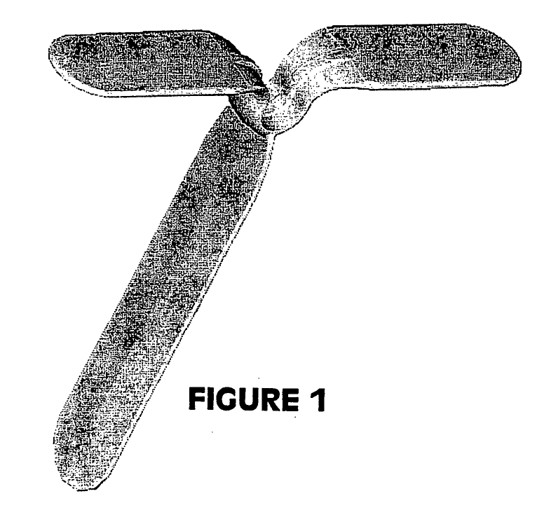

FIG. 1 is a perspective view of a preferred device in accordance with the present invention suitable for insertion in the bovine species, cow,

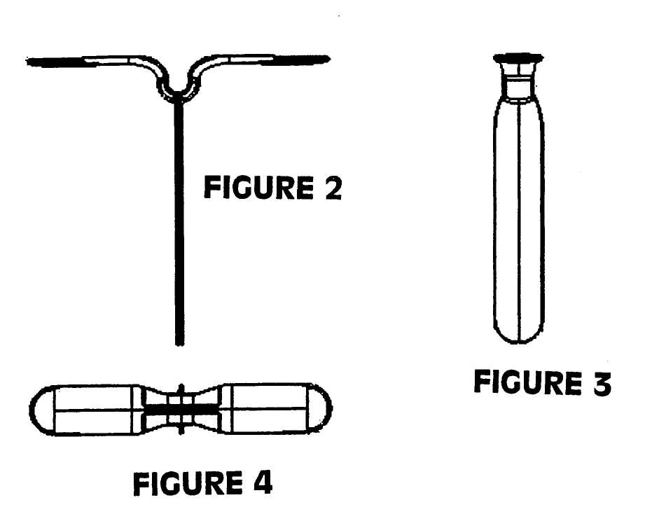

FIG. 2 is an elevation view of the device of FIG. 1,

FIG. 3 is another elevation view of the device of FIG. 1,

FIG. 4 is a plan view of the top of the hybrid “T” or substantially “T” or substantially “Y” form of the device of FIGS. 1 to 3,

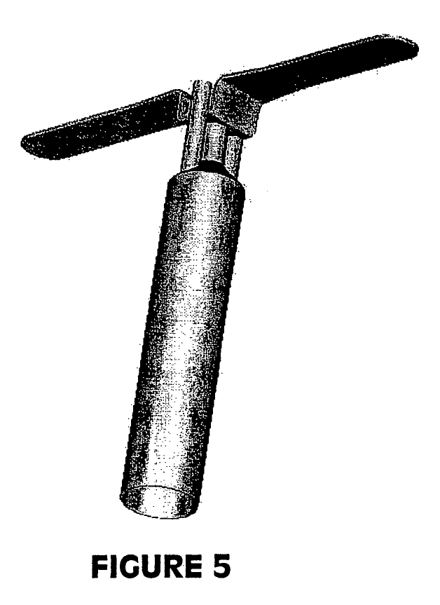

FIG. 5 is a perspective view of another preferred device in accordance with the present invention suitable for insertion in the bovine species, this variant having a tubular moulded body part adapted to receive as part of an assembly components to provide multiple active release (eg; using a mechanism of either our WO 99/29259 or PCT/NZ00/00155),

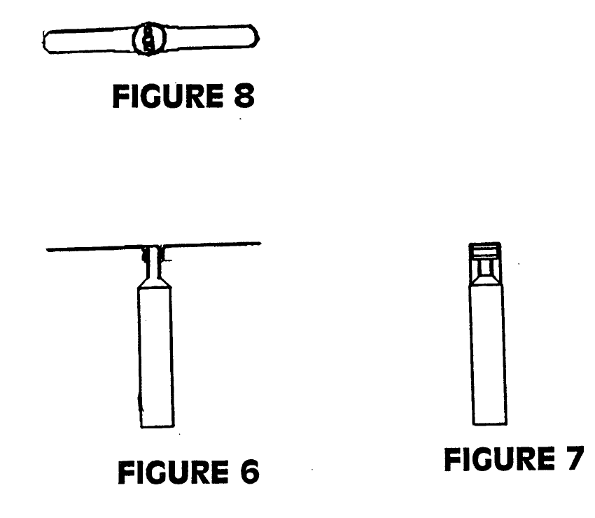

FIG. 6 is an elevation view of the device of FIG. 5,

FIG. 7 is another elevation view of the moulding of the device of FIG. 5,

FIG. 8 is a plan view of the top of the device of FIGS. 5 through 7, and

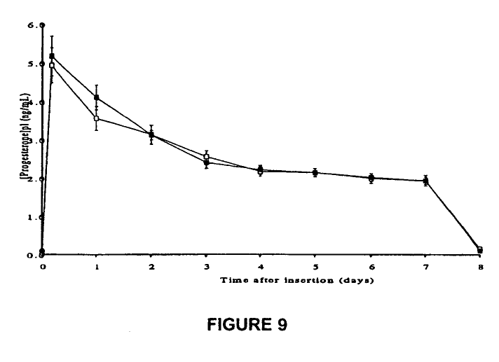

FIG. 9 is a plot of average progesterone level following insertion (ng/ml) over time (days) comparing a CIDR™ device against a device of the present invention as described in Study 1.

We have now established that intra vaginal devices mould using poly (ε-caprolactone) have been shown to possess retention characteristics superior to currently available intra vaginal inserts such as the CIDR™ 1900 (Pharmacia Animal Health, USA), see table 1.

TABLE 1
Number of animals treated for 8 days, number
retained and number lost using either a PCL
or CIDR 1900 ™ insert.

| | Number of animals | Number of inserts | Number of |
|---|---|---|---|
| Insert type | treated | retained for 8 days | inserts lost |
| PCL | 160 | 159 | 1 |
| CIDR 1900 ™ | 180 | 175 | 5 |

It has been identified that the parameter wing tension, which is a measure of the force (kg) required to bring the arms or projects of the insert from a relaxed state to a state wherein the projections form a 90° angle, remains relatively unchanged following an insertion period, see table 2.

TABLE 2
Wing tension of inserts prior to insertion for 8 days,
upon removal and % change using either
a PCL or CIDR 1900 ™ insert.

| | | Wing tension upon | Change in wing |
|---|---|---|---|
| | Initial wing | removal after 8 days | tension after 8 days |
| Insert type | tension (kg) | insertion (kg) | insertion (%) |
| PCL | 4.02 | 3.57 | 11* |
| CIDR 1900 ™ | 2.90 | 2.02 | 30 |

*not significantly different at p = 0.001

Materials

A suitable source of poly (ε-caprolactone) is that product TONE 767 (from Union Carbide Specialty Polymers and Products, Danbury, Conn., USA).

A suitable source of a starch-like polysaccharide is that product Mater-Bi™ (from Novamont, Italy).

Passive Release Devices

The device of FIGS. 1 through 4 include a body 1, two wings 2, the distal regions of which 3 extend outwardly yet are interconnected and are also connected to the body via a nodal region 4. In the plane of the drawing of FIG. 2 the nodal region is more bulky in the plane of the drawing in the preferred form than is the distal region of each wing thereby ensuring adequate control of the disposition of each wing relative to the body (limp through the body itself desirably is) yet providing a low trauma producing retention confirmation against the vaginal tract of the recipient animal.

A preferred content of the poly (ε-caprolactone) material is that disclosed in the aforementioned patent specification WO 99/63967 eg; includes 5 to 70% w/w hydroxypropyl β-cyclodextrin and 5 to 70% w/w of progesterone, the loading of progesterone being from 0.1 to 3 gms and with a matrix surface area of from 15 to 200 cm², the device enabling a blood serum level of greater than about 2 ng/ml of progesterone for a period of at least 5 days in the target species selected from cattle, sheep, goats, deer, etc.

### Study 1

Experimental Methods:

T-shaped poly (ε-caprolactone) intravaginal inserts were manufactured by injection moulding pre-extruded pellets of poly (ε-caprolactone) (Tone® P767, Union Carbide, USA) that contained 10% w/w progesterone (Micronised USP, Upjohn, USA).

The bioequivalence of the poly (ε-caprolactone) insert was determined by comparing its plasma levels to those of a commercially available reference product (CIDR™ intravaginal insert, Pharmacia Animal Health) using a cross-over experimental design in 12 ovariectomized cows. Plasma samples were collected immediately prior to insertion, every 24 hours after insertion, immediately prior to insert removal on day 7, and 24 hours following insert removal.

Progesterone content in the plasma was determined by direct radio immuno assay (Coat-a-Count, DPC, USA).

The poly (ε-caprolactone) insert was evaluated clinically over three separate rounds of treatment in a commercial dairy herd. Number of animals treated, number of inserts lost, number of cows submitted for artificial insemination, number of cows diagnosed as pregnant and conception rate were recorded.

Residual amounts of progesterone remaining in the inserts after administration were determined by Soxhlet extraction (CIDR™ inserts) or gravimetric analysis (poly (ε-caprolactone) inserts).

Results:

Upon insertion of the poly (ε-caprolactone) or CIDR™ intravaginal inserts plasma progesterone concentrations were observed to rise rapidly (FIG. 9). Elevations in plasma progesterone initially declined from approximately 5 ng/ml over the first 3 to 4 days and thereafter sustained constant concentrations of approximately 2 ng/ml to the seventh day. These concentrations had returned to undetectable basal levels within 24 hours of insert removal. Analysis of the pharmacokinetic parameters AUC, Cₘₐₓ and Tₘₐₓ showed that there were no significant differences between the test and reference product within or between rounds (Table 3). In addition, both inserts released the same amount of progesterone over the insertion period (Table 3).

The large scale clinical trial showed that poly (ε-caprolactone) insert retention was significantly greater compared to the CIDR insert. Of the 361 animals that received the poly (ε-caprolactone) insert only 1 was lost compared to 16 for the 387 animals that received the CIDR insert.

This study has shown that an intravaginal insert comprising poly (ε-caprolactone) and containing 10% w/w progesterone can be engineered to be bioequivalent to a commercially available intravaginal insert (CTDR). Large scale clinical trials of such a product show that the poly (ε-caprolactone) insert exhibited superior retention properties to the commercially available product (CIDR). This study has demonstrated that the biodegradable polymer poly (ε-caprolactone) is a suitable polymer with which to manufacture intravaginal inserts containing progesterone as an alternative to the current silicone based oestrous control products. See FIG. 9.

TABLE 3
AUC, Cₘₐₓ and Tₘₐₓ for test (poly (ε-caprolactone))and
reference (CIDR ™) intravaginal inserts.

| | Round 1 | | Round 2 | | Rounds 1 and 2 | |
|---|---|---|---|---|---|---|
| | poly | | poly | | poly | |
| Formulation | (ε-caprolactone) | CIDR | (ε-caprolactone) | CIDR | (ε-caprolactone) | CIDR |
| n | 6 | 6 | 6 | 6 | 12 | 12 |
| AUC (ng-day/mL) | 20.3 ± 1.2 | 19.2 ± 1.2 | 20.1 ± 1.5 | 20.3 ± 1.8 | 20.2 ± 0.9 | 19.7 ± 1.0 |
| Cₘₐₓ (ng/mL) | 6.2 ± 0.6 | 5.8 ± 0.4 | 4.6 ± 0.5 | 4.2 ± 0.6 | 5.3 ± 0.5 | 5.1 ± 0.4 |
| Tₘₐₓ (days) | 0.2 ± 0.0 | 0.2 ± 0.0 | 0.6 ± 0.2 | 1.0 ± 0.3 | 0.4 ± 0.1 | 0.6 ± 0.2 |
| Progesterone released (g) | 0.64 ± 0.0 | 0.60 ± 0.0 | 0.64 ± 0.0 | 0.56 ± 0.0 | 0.64 ± 0.0 | 0.58 ± 0.0 |

TABLE 4
Number of inserts retained, number of animals inseminated
and number of animals pregnant following treatment with the test
(poly (ε-caprolactone)) and reference (CIDR ™)
products over 3 rounds of treatment.

| | Trial I | Trial II | Trial III | Total |
|---|---|---|---|---|
| Treated (n) | | | | |
| poly (ε-caprolactone) | 143 | 167 | 51 | 361 |
| CIDR ™ | 137 | 152 | 98 | 387 |
| Total | 280 | 319 | 149 | 748 |
| Lost (n) | | | | |
| poly (ε-caprolactone) | 1 | 0 | 0 | 1 |
| CIDR ™ | 6 | 5 | 0 | 11 |

### Study 2

Experimental Methods:

T-shaped poly (ε-caprolactone) intravaginal inserts were manufactured by injection moulding pre-extruded pellets of poly (ε-caprolactone) (Tone® P767, Union Carbide, USA) with or with out the addition of 10% w/w progesterone (Micronised USP, Upjohn, USA).

The study was conducted using two rounds of 30 cows per round. Inserts were inserted into the vagina of the cows and removed 8 days later. Upon removal of the inserts at the completion of round 2 a veterinarian examined all cows.

Results:

No progesterone loaded poly (ε-caprolactone) intravaginal inserts were lost (54/54).

No progesterone blank poly (ε-caprolactone) intravaginal inserts were lost (6/6)

Examination by a veterinarian was conducted on each animal at the time of removal of the inserts at the completion of round 2 of the investigation; observations are presented in Table 5.

TABLE 5
All recorded observations upon removal of poly (ε-caprolactone) intravaginal inserts
at the end of 8 days insertion of round 2. Key 0 = good, 5 = severe abrasions.

| | | Left Wall | Right Wall | | Palpated/Vaginal |
|---|---|---|---|---|---|
| Insert | Cow | Condition | Condition | Comments | speculum |
| 1 | 37 | 0 | 0 | no indentations | Palpated |
| 2 | 7902 | 0 | 0 | no indentations | Palpated |
| 3 | 492 | 2 | 0 | no indentations, insert | Palpated/speculum |
| | | (abrasions @ | | sitting sideways causing | |
| | | 9 o'clock) | | vaginal irritation | |
| 4 | 8927 | 0 | 0 | no indentations | Palpated |
| 5 | 648 | 0 | 0 | no indentations | Palpated |
| 6 | 3786 | 0 | 0 | no indentations | Palpated |
| 7 | 8678 | 0 | 0 | no indentations, laying vertical | Palpated |
| 8 | 50 | 0 | 0 | no indentations | Palpated |
| 9 | 330 | 0 | 0 | no indentations, insert sitting | Palpated/speculum |
| | | | | vertically, stripy appearance, light | |
| | | | | irritation, photo taken | |
| 10 | 6930 | 0 | 0 | no indentations | Palpated/speculum |
| 11 | 8231 | 0 | 0 | no indentations, pinky mucus | Palpated |
| 12 | 57 | 0 | 0 | no indentations | Palpated |
| 13 | 7915 | 0 | 0 | no indentations | Palpated |
| 14 | 8942 | 0 | 0 | no indentations, pinky mucus | Palpated |
| 15 | 8611 | 0 | 2 | no indentations, rough tip to insert | Palpated |
| | | | (0.5 cm | caused irritation. | |
| | | | indentation 3 | | |
| | | | o'clock) | | |
| 16 | 8628 | 0 | 0 | no indentations | Palpated |
| 17 | 2 | 0 | 0 | no indentations | Palpated |
| 18 | 25 | 0 | 0 | no indentations | Palpated |
| 19 | 10 | 0 | 0 | no indentations | Speculum |
| 20 | 8643 | 0 | 0 | no indentations | Palpated |
| 21 | 8225 | 2 | 2 | no indentations, pinky mucus, | Palpated |
| | | | | insert turned round, insert | |
| | | | | discoloured pink with blood or | |
| | | | | fungus? Sounds bad but wasn't as | |
| | | | | all. | |
| 22 | 8680 | 2 | 0 | no indentations, pinky mucus, | Palpated |
| | | (1 cm | | rough end to insert. | |
| | | indentation @ | | | |
| | | 9 o'clock) | | | |
| 23 | 8647 | 0 | 0 | no indentations | Palpated |
| 24 | 7910 | 2 | 2 | no indentations, pinky mucus | Palpated |
| 25 | 8904 | 0 | 0 | no indentations | Speculum |
| 26 | 8654 | 0 | 0 | no indentations | Palpated |
| 27 | 366 | 0 | 0 | no indentations, pinky mucus | Speculum |
| 28 | 8644 | 0 | 0 | no indentations | Palpated |
| 29 | 8930 | 0 | 0 | no indentations | Speculum |
| 30 | 8652 | 0 | 0 | no indentations | Palpated |

What we claim is:

1. An intra vaginally retainable device which whilst at least in part limp in character is capable of being inserted in an insertion condition into the vaginal tract of a target species mammal, deploying resiliently to one or more retention condition(s) in such tract from the insertion condition and (if desired) being withdrawn from such tract, said device having
an elongate body, and
at least two wings capable of deploying to extend more outwardly from longitudinal axis of the body the constrained for insertion condition,
wherein said wings and said body have been formed from a material (impregnated or otherwise) having a resilient memory greater than that of nylon and which is substantially unaffected by the physiological conditions of the target species during such retention.

2. A device of claim 1 wherein said material has been impregnated with either a progesterone or a progesterone and clyclodextrin(s).

3. A device of claim 1 or 2 that has been moulded.

4. A device of any one of the preceding claims wherein said body is limp and said wings are limp and each is solely of said material (impregnated or otherwise) i.e. absent any spine or like structure of any other material.

5. A device of any one of the preceding claims wherein said material is a poly (ε-caprolactone) or a poly (ε-caprolactone) based matrix.

6. A device of any one of the preceding claims having a substantially “T” or “Y” configuration or a variant thereof which may include additional wings.

7. A device of claim 6 wherein the body (i.e. the stem of the substantially “T” or substantially “Y” configuration) is substantially limp from its nodal integrally moulded connection to its wings.

8. A device of claim 6 wherein said wings, when viewed in a direction that displays said substantially “T” or substantially “Y” configuration, are less bulky in their distal regions than their nodal region, (ie; their regions at and/or adjacent said nodal integrally moulded connection with said body).

9. An intravaginal device moulded in an at least progesterone impregnated poly (ε-caprolactone) material to define substantially a “T” (upper or lower case) shape or substantially a “Y” (upper or lower case) shape,
wherein said body defined by the stem of the shape is limp and at least the distal region of each arm of the shape is limp,
and wherein the poly (ε-caprolactone) material contains from 0.1 to 3 grams progesterone and the moulded material contains from 5 to 70% w/w progesterone and from 0 to 70% cyclodextrine,
and wherein the moulded surface is from 15 to 200 cm².

10. A device of claim 9 wherein each arm has a distal region extending outwardly from a curved proximal region that outstands from the stem.

11. A device of claim 10 wherein the curved proximal region of one arm is similar to that of the other and each curve is in plane in which its distal regions has a favoured freedom of resilient deformation.

12. A device of claim 11 wherein said curve is an integral “U” shape at one end of the stem, from which U shape each arm continues integrally to extend outwardly of the stem axis thereby to define substantially an upper case “T” shape with the stem, the “U” region being a nodal zone of the retention zone of each wing from the stem.

13. A device of any one of claims 9 to 12 wherein the stem is from 50 to 150 mms in length.

14. An intra vaginal device of a moulded matrix which includes a material capable of having or having a resilience memory better than that of nylon, said device having an elongate limp body from which at least a pair of retention wings depend via a nodal region, said nodal region controlling the planar flexure of proximate regions of each wing more strongly than the planar flexure of distal regions of each wing, such planar flexure being in a plane that can include substantially all of the elongate body.

15. A device of claim 14, wherein in a relaxed condition with said body substantially straight, the angle to the distal ends of each wing from the longitudinal axis of the body from the non nodal region end is less than 150 degrees.

16. A device of claim 15, wherein intra vaginally in a retention condition said angle is in the range of from 90 to 180 degrees.

17. A device of any one of claims 14 to 16 wherein the length of the body is from 50 to 150 mms.

18. A device of any one of claims 14 to 17 wherein at least in part the moulded matrix is of material which includes therein both a clyclodextrin and an intra vaginally effective active agent.

19. A device of any one of claims 14 to 18 wherein, is at least in part, the device is of a polymer or an impregnated polymer.

20. A device of claim 19 said polymer is poly (ε-caprolactone).

21. A device of any one of claims 14 to 20 wherein the clyclodextrin(s) comprise from 5 to 70% w/w of the matrix.

22. A device of any one of claims 14 to 21 wherein an intravaginally effective active agent(s) comprises from 5 to 70% w/w of the matrix.

23. A device of claim 22 wherein said agent is progesterone in the concentration of 5 to 70% w/w of the matrix.

24. A device of any one of claims 14 to 23 wherein hydroxypropyl β-clyclodextrin in the concentration of 5 to 70% w/w is present in the matrix.

25. A device of any one of claims 14 to 24 capable of achieving within a target animal a blood serum level of greater than about 2 ng/ml for a period of at least 5 days of progesterone solely as a result of inserting and retaining in the animal for at least the 5 day period.

26. A device of claim 25 having a loading of from 0.1 to 3 gms of progesterone.

27. A device of any one of claims 14 to 26 wherein said device has an impregnated matrix has a surface of from 15 to 200 cm².

28. An intra vaginal device of a kind capable (at least) of
(i) being inserted into the vaginal tract of a target species mammal, and
(ii) deploying resiliently to one or more retention condition(s) in such tract,
wherein in said retention condition(s) at least two wings will extend more outwardly from the vaginal tract axis than in the insertion condition,
and wherein the wings and at least part of the body of the device from which the wings depend is moulded in a poly (ε-caprolactone) material (impregnated or otherwise),
and wherein said wings are integral with said at least part of the body of the device through a nodal transition of greater rigidity owing to bulk than the distal region(s) of each wing.

29. A intra vaginal device of a kind capable of being inserted into the vaginal tract of a target species mammal, deploying resiliently to one or more retention condition(s) in such tract, and (if desired) being withdrawn from such tract,
wherein at least two wings depend from at least part of the body of the device, said wings and said at least part of the body of the device being a unitary moulding of a suitable resilient material,
and wherein the moulded form of said wings and at least part of the body is such that a more distal region of the wings is more flexible in bending towards one or both of the vaginal tract axis and the body axis (if any) than the body proximal “nodal region” of said wings with said at least part of the body.

30. A device of claim 29 which has been coated in order to minimise physiological effects on the integrity of such material during vaginal tract retention.

31. A device of claim 29 or 30 wherein said material is a suitable poly (ε-caprolactone) material.

32. A device of any one of claims 29 to 31 which is substantially “T” or “Y” shaped with a nodal region which includes the body proximate regions of each wing and the wing proximate region of at least part of the body (i.e. the body being the stem of the “T” or “Y”), such node whilst of a similar (if not identical) material to the distal region of each wing being less flexible owing to greater moulded bulk.

33. A device of claim 32 wherein the distal region of the wings is thinner in at least a plane which better enables bending in either rotational sense relative to the vaginal tract axis and/or a substantially “T” or “Y” stem defined body of the device.

34. A device of any one of claims 29 to 33 wherein the body of the device is fully moulded.

35. A device of claim 34 wherein the moulding is of poly (ε-caprolactone) impregnated with material(s) to be released.

36. A device of claim 35 wherein the material(s) to be released is progesterone or progesterone and clyclodextrin.

37. An intra vaginal device of a kind capable of being inserted into the vaginal tract of a target species mammal, deploying resiliently to one or more retention condition(s) in such tract to release progesterone, and being withdrawn from such tract,
wherein said retention condition(s) at least two wings extend more outwardly from the vaginal tract axis than in the insertion and withdrawal conditions,
wherein the wings and the body of the device from which the wings depend is moulded in poly (ε-caprolactone) impregnated with progesterone(s) (and optionally also clyclodextrin(s)) to a substantially limp elongate body form bulkily (at least in one plane) connected to the wings at the nodal region with the wings rendered more flexible (preferably in that plane) in their non-nodal region or regions to better conform to the wall of the vaginal tract.

38. An intra vaginal device moulded to substantially a “T” or substantially “Y” configuration where the wings (those parts that extend outwardly from the stem of the substantially “T” or substantially “Y” configuration) each have the nodal region (ie; the transition from the stem to the wings) more bulky in a plane than is the distal region of each wing, thereby to ensure as far as flexibility of each wing in total is concerned greater flexibility over outer regions of each wing than at the nodal region.

39. A device of claim 38 of a form where the transition from the stem to the wings is a substantially “U” form blended into otherwise outstanding arms of a “T” wings.

40. A device of claim 38 and 39 which has a wing span of from 125 to 175 mm.

41. A device of any one of claims 38 to 40 wherein each wing is wider (i.e. of greater dimension in a plane normal to the plane of the “T” or substantially “T” form) than it is in the plane of the “T”.

42. A device of any one of claims 38 to 41 where said substantially “T” or substantially “Y” form has a body (i.e. the stem) that is substantially of a similar configuration and a similar order of flexibility to the distal region or regions of each wing (at least away from the nodal region).

43. A passive or active release device for a medicament or other substance for a target species mammal, said device comprising a device of a kind capable of being inserted into a body cavity of a target species mammal and deploying resiliently to one or more retention condition(s) wherein said device is wholly or in part moulded from poly (ε-caprolactone) (optionally impregnated with at least a physiologically effective agent to be released there from or optionally to carry some device or material to be body cavity retained at least for a period) and wherein the device in its moulded or moulded and optional assembled form has a plurality of wings (ie; two or more) each wing connected to the other through a nodal region and each also connected to at least part of the body of the device by the same nodal region, such nodal region owing to its bulk in section in at least one plane (poly (ε-caprolactone) alone or impregnated poly (ε-caprolactone)) having less flexibility than more distal regions or a more distal region of the wings or each wing.

44. A device of claim 43 wherein the flexibility of the distal region or regions is further enhanced by (i) the asymmetry of the cross-section of such distal region or regions of the wings and/or (ii) the asymmetry of the cross-section of the nodal region.

45. A device substantially as hereinbefore described with reference to any one or more of the accompanying drawings.

46. A method of retaining a physiologically effective sensor and/or physiologically effective agent delivery device in a body cavity of a target species mammal reliant on the use of a device of any one of the preceding claims.

47. The use of a device of any one of claims 1 to 45.

48. The use of claim 47 for herd oestrus synchrony purposes.
